(19) United States
(12) Patent Application Publication
(10) Pub. No.: US 2024/0084049 A1
(43) Pub. Date: Mar. 14, 2024

(54) THREE-DIMENSIONAL CONTROL CATALYST USED IN RADICAL POLYMERIZATION, POLYMER PRODUCTION METHOD, AND ACRYLIC POLYMER

(71) Applicant: Kyoto University, Kyoto (JP)

(72) Inventor: Shigeru Yamago, Kyoto (JP)

(73) Assignee: Kyoto University, Kyoto (JP)

(21) Appl. No.: 17/642,067

(22) PCT Filed: Sep. 9, 2020
(86) PCT No.: PCT/JP2020/034144

§ 371 Date: Mar. 10, 2022

(30) Foreign Application Priority Data
Sep. 10, 2019 (JP) 2019-164905

(51) Int. Cl. C08F 4/12; C08F 120/06

(52) U.S. Cl. CPC C08F 4/12; C08F 120/06; C08F 2438/00

(57) ABSTRACT

The present invention provides a stereocontrol catalyst for use in radical polymerization that is applicable to polymerization of a broad range of monomers and that enables polymerization with control of both molecular weight (molecular weight distribution) and stereoselectivity, a method for producing a polymer using the stereocontrol catalyst for use in radical polymerization, and an acrylic polymer. Provided is a stereocontrol catalyst for use in radical polymerization, containing: a rare-earth metal salt compound; and a hydroxy group-containing compound.

### TECHNICAL FIELD

The present invention relates to a stereocontrol catalyst for use in radical polymerization, a method for producing a polymer, and an acrylic polymer.

### BACKGROUND ART

Various polymers have been produced by radical polymerization on an industrial scale for a long time. This conventional production method has difficulty in producing polymers with desired molecular weight distributions because propagating radicals in the series of radical polymerization reactions are inactivated by termination reactions such as recombination and disproportionation or by side reactions such as chain transfer, causing the resulting polymers to have broad molecular weight distributions. The conventional production method also has difficulty in controlling the stereoselectivity of the resulting polymers.

Non-Patent Literature 1, for example, proposes a method for reversible addition-fragmentation chain transfer (RAFT) polymerization of N-isopropylacrylamide under the presence and absence of Y(OTf)₃, a Lewis acid. Non-Patent Literature 1 teaches that this method can control the molecular weight and the stereoselectivity of the resulting polymer.

Meanwhile, the living radical polymerization method has attracted attention as a way to solve the technical problems with the conventional radical polymerization method. Living radical polymerization is believed to enable control of molecular weight, molecular weight distribution, molecular structure, and the like as well as production of block polymers. Many studies have been made particularly on monomers such as styrene and (meth)acrylate, and it is known that the polymers of these monomers can be controlled in their primary structure with a certain level of precision.

### CITATION LIST

### Non-Patent Literature

Non-Patent Literature 1: Macromolecules 2004, 37, 1702-1710

### SUMMARY OF INVENTION

### Technical Problem

However, the method of Non-Patent Literature 1 may not always be applicable to production of desired polymers because monomers polymerizable by this method are limited to those soluble in alcohol.

Moreover, applying the method of Non-Patent Literature 1 to the living radical polymerization method may impair living radical characteristics, making it difficult to control the molecular weight, the molecular weight distribution, and the stereoselectivity of the resulting polymer.

The present invention aims to provide a stereocontrol catalyst for use in radical polymerization that is applicable to polymerization of a broad range of monomers and that enables polymerization with control of both molecular weight (molecular weight distribution) and stereoselectivity, a method for producing a polymer using the stereocontrol catalyst for use in radical polymerization, and an acrylic polymer.

### Solution to Problem

The gist of the present invention is as follows.

(1) A stereocontrol catalyst for use in radical polymerization, containing a rare-earth metal salt compound and a hydroxy group-containing compound.

(2) The stereocontrol catalyst for use in radical polymerization according to (1), wherein the rare-earth metal salt compound is a rare-earth metal trifluoromethanesulfonate.

(3) The stereocontrol catalyst for use in radical polymerization according to claim (1) or (2), wherein the rare-earth metal salt compound is a salt of a trivalent rare-earth metal.

(4) The stereocontrol catalyst for use in radical polymerization according to any one of (1) to (3), wherein the hydroxy group-containing compound is an alcoholic compound.

(5) The stereocontrol catalyst for use in radical polymerization according to any one of (1) to (4), wherein the hydroxy group-containing compound is a binaphthol derivative.

(6) A method for producing a polymer, including polymerizing a (meth)acrylic monomer under the presence of the stereocontrol catalyst for use in radical polymerization according to any one of (1) to (5).

(7) The method for producing a polymer according to claim (6), wherein the (meth)acrylic monomer is polymerized under the presence of the stereocontrol catalyst for use in radical polymerization in a solvent that contains a compound different from the hydroxy group-containing compound contained in the stereocontrol catalyst for use in radical polymerization.

(8) The method for producing a polymer according to claim (6) or (7), which is performed by living radical polymerization.

(9) An acrylic polymer containing, in a molecule, a meso form and a racemo form, the acrylic polymer having a percentage of the meso form of 65% or more and a molecular weight distribution of 1.8 or less.

The present invention is described in detail below.

The stereocontrol catalyst for use in radical polymerization of the present invention contains a rare-earth metal salt compound.

The rare-earth metal salt compound functions as a Lewis acid.

The rare-earth metal salt compound is not limited as long as it has a rare-earth metal and a counterion component of the rare-earth metal.

Examples of the rare-earth metal include scandium, yttrium, and lanthanide metals (lanthanum, cerium, praseodymium, neodymium, promethium, samarium, europium, gadolinium, terbium, dysprosium, holmium, erbium, thulium, ytterbium, and lutetium). In the present invention, particularly the use of a lanthanide metal may allow greater control of molecular weight (molecular weight distribution) and stereoselectivity.

Preferred counterion components of the rare-earth metal include trifluoromethanesulfonic acid and trifluoromethanesulfonylimide. Trifluoromethanesulfonic acid is particularly preferably used.

Examples of the rare-earth metal salt compound include rare-earth metal trifluoromethanesulfonates such as ytterbium trifluoromethanesulfonate [Yb(OTf)₃], yttrium trifluoromethanesulfonate [Y(OTf)₃], scandium trifluoromethanesulfonate, erbium trifluoromethanesulfonate, europium trifluoromethanesulfonate, and lutetium trifluoromethanesulfonate and trifluoromethanesulfonimide rare-earth metal salts such as trifluoromethanesulfonimide yttrium [Y(NTf₂)₃]. Preferred among these are rare-earth metal trifluoromethanesulfonates.

The rare-earth metal salt compound is preferably a salt of a trivalent rare-earth metal.

These rare-earth metal salt compounds may be used alone or in combination of two or more thereof.

The amount of the rare-earth metal salt compound in the stereocontrol catalyst for use in radical polymerization of the present invention is appropriately determined according to factors such as the monomer type and the molecular weight of a target polymer. The amount is preferably 2 to 70 mol %, more preferably 5 to 40 mol %, still more preferably 10 to 30 mol %.

The stereocontrol catalyst for use in radical polymerization of the present invention contains a hydroxy group-containing compound.

The hydroxy group-containing compound functions as a ligand. The hydroxy group-containing compound is not limited as long as it has a hydroxy group. The hydroxy group-containing compound used may be halogen-substituted, for example chlorine- or bromine-substituted.

Examples of the hydroxy group-containing compound include water, alcoholic compounds, phenols, and dihydric to octahydric polyphenols. The hydroxy group-containing compound is preferably water or an alcoholic compound.

Examples of the alcoholic compound include monohydric alcohols such as methanol, ethanol, n-propanol, isopropanol, n-butanol, sec-butanol, tert-butanol, isobutanol, octanol, and 2-methoxyethanol; dihydric alcohols such as ethylene glycol, propylene glycol, 1,3-butylene glycol, 1,4-butanediol, 1,6-hexanediol, 3-methylpentanediol, diethylene glycol, triethylene glycol, tetraethylene glycol, neopentyl glycol, 1,4-bis (hydroxymethyl)cyclohexane, 1,4-bis(hydroxyethyl)benzene, and 2,2-bis(4,4′-hydroxycyclohexyl)propane; trihydric alcohols such as glycerol and trimethylolpropane; tetrahydric to octahydric alcohols such as pentaerythritol, diglycerol, α-methyl glucoside, sorbitol, xylitol, mannitol, dipentaerythritol, glucose, fructose, and sucrose. The hydroxy group of the alcoholic compound may be any of primary to tertiary hydroxy groups. The alcoholic compound preferably has a primary or secondary hydroxy group, more preferably a primary hydroxy group, so as to prevent substituents other than the hydroxy group from causing steric hindrance. In a polyhydric alcohol, at least two hydroxy groups are preferably primary hydroxy groups. The alcoholic compound is preferably an alcohol having a carbon number of 2 or greater. The alcoholic compound is also preferably an alcohol having two or more hydroxy groups. In this case, two hydroxy groups are preferably end groups.

Examples of the phenols include phenol and cresol. Examples of the polyphenols include pyrogallol, catechol, and hydroquinone.

Other hydroxy group-containing compounds that can be used include bisphenols such as bisphenol A, bisphenol F, and bisphenol S; polybutadiene polyols; castor oil polyols; and (co)polymers of hydroxyalkyl (meth)acrylates.

The hydroxy group-containing compound is preferably a polyalkylene glycol compound.

Examples of the polyalkylene glycol compound include diethylene glycol, triethylene glycol, tetraethylene glycol, pentaethylene glycol, hexaethylene glycol, diisopropylene glycol, dipropylene glycol, tripropylene glycol, and tetrapropylene glycol. The polyalkylene glycol compound is preferably diethylene glycol or triethylene glycol.

In the present invention, the hydroxy group-containing compound is preferably a binaphthol derivative having a hydroxy group.

The binaphthol derivative having a hydroxy group is preferably one in which a hydroxy group is directly bonded to a binaphthyl group, or a compound having a polyalkylene glycol chain.

For example, the binaphthol derivative having a hydroxy group is preferably a binaphthol derivative represented by the following formula (1). In particular, the binaphthol derivative having a hydroxy group is more preferably a binaphthol derivative represented by the following formula (2).

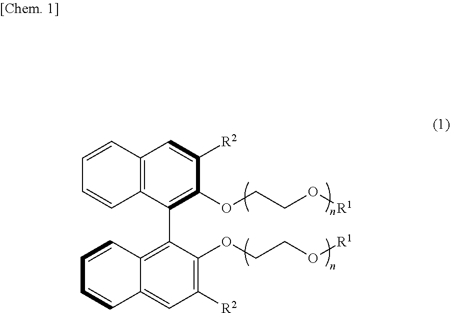

In the formula (1), R¹ represents a hydrogen atom, R² represents a hydrogen atom or a substituent, and n represents an integer of 0 or greater. The substituent is preferably an alkyl group or an aromatic group. Moreover, n is preferably an integer of 0 to 3.

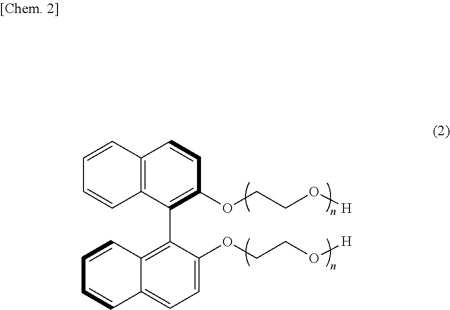

In the formula (2), n represents an integer of 0 or greater. Here, n is preferably an integer of 0 to 3.

The binaphthol derivative having a hydroxy group includes stereoisomers having different configurations (R isomer, S isomer) and racemates.

The binaphthol derivative having a hydroxy group may be one in which some of the unsaturated bonds of the binaphthol structure are saturated bonds (e.g., a binaphthol derivative represented by the following formula (3)).

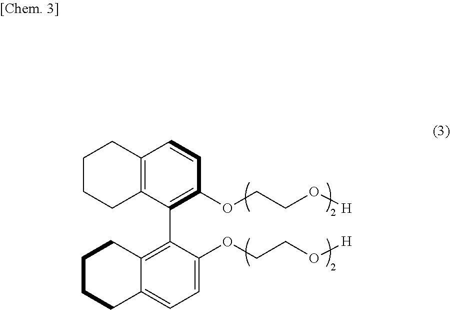

The amount of the stereocontrol catalyst for use in radical polymerization of the present invention is appropriately determined according to factors such as the monomer type and the molecular weight of a target polymer. The amount is preferably 5 to 100 mol % relative to a monomer, more preferably 10 to 30 mol % relative to a monomer. The amount of the stereocontrol catalyst is still more preferably 10 to 20 mol % relative to a monomer.

The amount of the hydroxy group-containing compound in the stereocontrol catalyst for use in radical polymerization is preferably 30 to 95 mol %, more preferably 40 to 90 mol %, still more preferably 50 to 80 mol %.

The stereocontrol catalyst for use in radical polymerization of the present invention contains the rare-earth metal salt compound and the hydroxy group-containing compound, so that the rare-earth metal salt compound and the hydroxy group-containing compound coordinate to form a complex.

The molar ratio of the rare-earth metal salt compound to the hydroxy group-containing compound (rare-earth metal salt compound/hydroxy group-containing compound) is preferably within the range of 0.01 to 20, more preferably 0.1 to 20. The molar ratio is still more preferably within the range of 0.5 to 10. When water or methanol is used as the hydroxy group-containing compound, the molar ratio of the rare-earth metal salt compound to the hydroxy group-containing compound is particularly preferably at least 0.01.

Here, the stereocontrol catalyst for use in radical polymerization of the present invention may contain compounds other than the rare-earth metal salt compound and the hydroxy group-containing compound, such as unavoidable impurities.

The stereocontrol catalyst for use in radical polymerization of the present invention can be used for any monomer material. Monomer material have sometimes been limited to those soluble in alcohol, but the stereocontrol catalyst for use in radical polymerization of the present invention enables the use of monomers insoluble in alcohol.

The monomer material may preferably be a (meth)acrylic monomer, for example.

The (meth)acrylic monomer refers to an acrylic monomer or a methacrylic monomer.

Examples of preferred (meth)acrylic monomers include amide group-containing (meth)acrylates; alkyl (meth)acrylates such as methyl (meth)acrylate, ethyl (meth)acrylate, propyl (meth)acrylate, and butyl (meth)acrylate; carboxy group-containing (meth)acrylates such as (meth)acrylic acid; hydroxy group-containing (meth)acrylates such as 2-hydroxyethyl (meth)acrylate, 2-hydroxybutyl (meth)acrylate, and 2-hydroxybutyl (meth)acrylate; and fluorine-containing (meth)acrylates such as 2,2,2-trifluoroethyl methacrylate, 2-(perfluorobutyl)ethyl methacrylate, 2-(perfluorohexyl)ethyl methacrylate, and 2-(perfluorobutyl)ethyl methacrylate. Preferred among these are amide group-containing (meth) acrylates.

Examples of the amide group-containing (meth)acrylates include acrylamide, N,N-dimethylacrylamide, N,N-diethylacrylamide, N-methylacrylamide, N-ethylacrylamide, N-isopropylacrylamide, N-cyclopropylacrylamide, N,N-dimethylaminopropylacrylamide, N,N-diethylaminopropylacrylamide, acryloylmorpholine, methacrylamide, N,N-dimethylmethacrylamide, N,N-diethylmethacrylamide, N-methylmethacrylamide, N-ethylmethacrylamide, N-isopropylmethacrylamide, N-cyclopropylmethacrylamide, N,N-dimethylaminopropylmethacrylamide, and N,N-diethylaminopropylmethacrylamide.

Preferred among these are amide group-containing acrylates having a tertiary amide group such as N,N-dimethylacrylamide and N,N-diethylacrylamide and amide group-containing acrylates having a secondary amide group such as N-methylacrylamide, N-ethylacrylamide, and N-isopropylacrylamide.

In the amide group-containing (meth)acrylates, the nitrogen of the amide group may form part of a cyclic structure. Examples of the amide group-containing (meth)acrylates include 1-(1-oxo-2propenyl)pyrrolidine, 1-(1-oxo-2propenyl)piperidine, 1-(1-oxo-2propenyl)azepane, and 1-(1-oxo-2propenyl)piperazine.

Other monomer materials that can be used include vinyl esters such as vinyl acetate, vinyl pivalate, vinyl formate, vinyl propionate, vinyl butyrate, vinyl n-caproate, vinyl isocaproate, vinyl octanoate, vinyl laurate, vinyl palmitate, vinyl stearate, vinyl trimethylacetate, vinyl chloroacetate, vinyl trichloroacetate, and vinyl trifluoroacetate; vinyl ethers such as methyl vinyl ether, ethyl vinyl ether, and butyl vinyl ether; and vinyl ketones such as methyl vinyl ketone, ethyl vinyl ketone, butyl vinyl ketone, hexyl vinyl ketone, and isopropenyl vinyl ketone.

These vinyl monomers may be used alone or in combination of two or more thereof.

The method for producing a polymer of the present invention includes polymerizing a (meth)acrylic monomer under the presence of the stereocontrol catalyst for use in radical polymerization described above. The method thus is applicable to polymerization of a broad range of monomers and enables polymerization with control of both molecular weight (molecular weight distribution) and stereoselectivity.

In particular, performing the method for producing a polymer of the present invention by living radical polymerization allows more suitable control of molecular weight (molecular weight distribution) and stereoselectivity without impairing living radical characteristics.

In the method for producing a polymer of the present invention, a radical generator may be added. Examples of radical generators that may be used include azo compounds, organic peroxides, and non-polar radical generators.

Specific examples of the azo compounds include 2,2′-azobisisobutyronitrile, 2,2′-azobis-2-methylbutyronitrile, 2,2′-azobis-2,4-dimethylvaleronitrile, 1,1′-azobis-1-cyclohexanecarbonitrile, dimethyl-2,2′-azobisisobutyrate, 4,4′-azobis-4-cyanovaleric acid, 2,2′-azobis-(2-amidinopropane)dihydrochloride, 2,2′-azobis(4-methoxy-2,4-dimethylvaleronitrile), and 1-((1-cyano-1-methylethyl)azo)formamide.

Specific examples of the organic peroxides include hydroperoxides such as t-butyl hydroperoxide, p-menthane hydroperoxide, and cumene hydroperoxide; dialkyl peroxides such as dicumyl peroxide, t-butyl cumyl peroxide, α,α′-bis (t-butylperoxy-m-isopropyl)benzene, di-t-butyl peroxide, 2,5-dimethyl-2,5-di(t-butylperoxy)-3-hexyne, and 2,5-dimethyl-2,5-di(t-butylperoxy)hexane; diacyl peroxides such as dipropionyl peroxide and benzoyl peroxide; peroxyketals such as 2,2-di(t-butylperoxy)butane, 1,1-di(t-hexylperoxy)cyclohexane, 1,1-di(t-butylperoxy)-2-methylcyclohexane, and 1,1-di(t-butylperoxy)cyclohexane; peroxy esters such as t-butyl peroxyacetate and t-butyl peroxybenzoate; peroxycarbonates such as t-butyl peroxyisopropyl carbonate and di(isopropylperoxy)dicarbonate; alkyl silyl peroxides such as t-butyl trimethylsilyl peroxide; and cyclic peroxides such as 3,3,5,7,7-pentamethyl-1,2,4-trioxepane, 3,6,9-triethyl-3,6,9-trimethyl-1,4,7-triperoxonane, and 3,6-diethyl-3,6-dimethyl-1,2,4,5-tetroxane.

Specific examples of the non-polar radical generators include 2,3-dimethyl-2,3-diphenylbutane, 3,4-dimethyl-3,4-diphenylhexane, 1,1,2-triphenylethane, and 1,1,1-triphenyl-2-phenylethane. These radical generators may be used alone or in combination of two or more thereof.

When the method for producing a polymer of the present invention is performed by living radical polymerization, a compound usually used as a chain transfer agent may be used as a chain transfer agent. For example, the chain transfer agent may be any of catalysts used for nitroxide-mediated polymerization (NMP), catalysts used for atom transfer radical polymerization (ATRP), catalysts used for reversible addition-fragmentation chain transfer (RAFT) polymerization, catalysts used for reversible-deactivation radical polymerization (TERP), and catalysts used for reversible complexation mediated polymerization (RCMP). The chain transfer agent is preferably an organotellurium compound.

For example, the organotellurium compound is preferably a compound represented by the following formula (4), more preferably a compound represented by the following formula (5). The chain transfer agent may also serve as a radical generator.

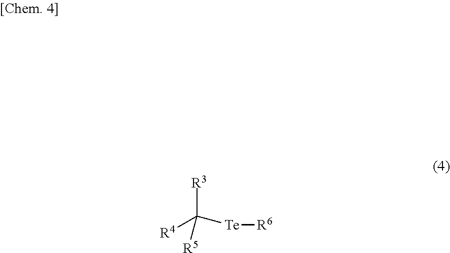

In the formula (4), R³ represents an alkoxycarbonyl group, an acyl group, an amide group, an aryl group, a substituted aryl group, an aromatic heterocyclic group, or a cyano group, R⁴ and R⁵ each independently represent a hydrogen atom or a C1-C8 alkyl group, and R⁶ represents a C1-C8 alkyl group, an aryl group, a substituted aryl group, or an aromatic heterocyclic group.

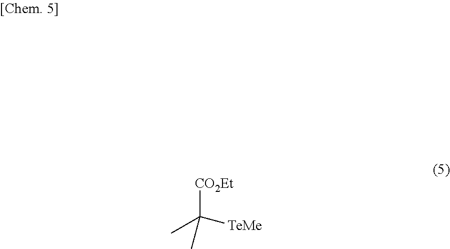

The polymerization method used for the method for producing a polymer of the present invention may be a conventionally known method. Examples include a bulk polymerization method, a solution polymerization method, a suspension polymerization method, and an emulsion polymerization method. In order to reduce chain transfer and more precisely control polymerization, a solution polymerization method with very high monomer concentration or a bulk polymerization method is suitably used.

Examples of a solvent that can be used in the method for producing a polymer of the present invention include, but not limited to, water; hydrocarbon solvents such as benzene and toluene; ether solvents such as diethyl ether, tetrahydrofuran, diphenyl ether, anisole, and dimethoxybenzene; halogenated hydrocarbon solvents such as dichloromethane, chloroform, and chlorobenzene; ketone solvents such as acetone, methyl ethyl ketone, and methyl isobutyl ketone; alcoholic solvents such as methanol, ethanol, propanol, isopropanol, n-butyl alcohol, and tert-butyl alcohol; nitrile solvents such as acetonitrile, propionitrile, and benzonitrile; acetate solvents such as ethyl acetate and butyl acetate; cyclic ester solvents such as γ-butyrolactone and ε-caprolactone; cyclic carbonate solvents such as ethylene carbonate and propylene carbonate; amide solvents such as N,N-dimethylformamide and N-methylpyrrolidone; and aprotic polar solvents such as dimethyl sulfoxide. The solvent preferably contains a compound different from the hydroxy group-containing compound in the stereocontrol catalyst for use in radical polymerization, more preferably contains a compound not containing a hydroxy group, still more preferably a halogenated hydrocarbon solvent.

For initiation of the polymerization reaction in the method for producing a polymer of the present invention, preferably, a mixture containing the stereocontrol catalyst for use in radical polymerization, the (meth)acrylic monomer, the solvent, and the polymerization initiator (radical generator) is prepared in a reaction vessel in an inert gas atmosphere such as a nitrogen atmosphere, and subjected to polymerization reaction. If necessary, the chain transfer agent may be added to the reaction vessel together with or instead of the radical generator.

In the method for producing a polymer of the present invention, the amount of the stereocontrol catalyst for use in radical polymerization added is preferably 5 to 50 mol %, more preferably 10 to 30 mol %, relative to the (meth)acrylic monomer.

In the method for producing a polymer of the present invention, the polymerization temperature is preferably −70° C. to 100° C., more preferably 0° C. to 30° C.

The polymerization time is not limited, and may be the time required for the monomer material (e.g., (meth)acrylic monomer) to be consumed.

The pressure during polymerization is not limited, and may be around atmospheric pressure.

In the method for producing a polymer of the present invention, light irradiation is preferably performed in polymerization reaction.

Examples of a light source for the light irradiation include mercury lamps and LEDs.

After completion of the polymerization, for example, the polymerization reaction system may be cooled to 0° C. or lower or a polymerization inhibitor may be added, so as to terminate the polymerization reaction. The polymer produced is then recovered by a conventional method. For example, the polymer solution may be diluted in a solvent such as toluene as needed and then put in methanol. The resulting precipitate may be washed several times and dried at room temperature under reduced pressure, whereby a target polymer is obtained.

Preferably, the acrylic polymer of the present invention contains a meso form and a racemo form and has a percentage of the meso form of 65% or more and a molecular weight distribution of 1.8 or less. The acrylic polymer preferably has a percentage of the meso form of 75% or more, more preferably 85% or more, still more preferably 90% or more. The acrylic polymer preferably has a molecular weight distribution of 1.6 or less. Particularly in radical polymerization using a chain transfer agent (living radical polymerization), the acrylic polymer preferably has a molecular weight distribution of 1.5 or less, more preferably 1.2 or less.

The polymer of the present invention has a controlled molecular weight (molecular weight distribution) and controlled stereoselectivity, so that it can be suitably applied to elastomers, for example. The polymer is particularly promising for application to biomaterials and the like because the polymer can be applied to amide group-containing acrylic resins, which are highly biocompatible. The physical properties of the polymer can be adjusted (e.g., the crystallinity of the polymer can be improved) by controlling the stereoselectivity (e.g., by increasing the percentage of the meso form).

The acrylic polymer of the present invention can be suitably prepared by the method for producing a polymer of the present invention, and more suitably prepared by living radical polymerization.

### Advantageous Effects of Invention

The present invention can provide a stereocontrol catalyst for use in radical polymerization that is applicable to polymerization of a broad range of monomers and that enables polymerization with control of both molecular weight (molecular weight distribution) and stereoselectivity, a method for producing a polymer using the stereocontrol catalyst for use in radical polymerization, and an acrylic polymer.

Particularly when performed by living radical polymerization, the stereocontrol catalyst for use in radical polymerization of the present invention enables more suitable control of molecular weight (molecular weight distribution) and stereoselectivity without impairing living radical characteristics.

Moreover, the stereocontrol catalyst for use in radical polymerization of the present invention enables sufficient control of molecular weight (molecular weight distribution) and stereoselectivity even with a short polymerization time.

### DESCRIPTION OF EMBODIMENTS

In the following, the present invention is described in more detail with reference to examples. The present invention should not be limited to these examples.

### Example 1

### [Radical Polymerization]

In a nitrogen atmosphere, a glass reaction container was charged with 1.1 mL of dichloromethane as a solvent, 0.57 mmol of N,N-diethylacrylamide (DEAA) as a monomer, 0.23 mmol (0.20 equivalents) of ytterbium trifluoromethanesulfonate [Yb(OTf)₃] as a Lewis acid, 0.23 mmol (0.20 equivalents) of water as a ligand, and 0.017 mmol (0.03 equivalents) of azobisisobutyronitrile [AIBN] as an initiator. They were then uniformly mixed to prepare a polymerization reaction solution. The obtained polymerization reaction solution was polymerized at 0° C. for 14 hours while being irradiated with a 500-W mercury lamp, whereby a polymer was obtained.

### Examples 2 to 15 and Comparative Examples 1 to 5

Polymers were obtained by polymerization as in Example 1, except that the polymerization conditions (temperature and time) were as shown in Table 1 and the types and amounts (equivalents) of monomer, Lewis acid, and ligand were as shown in Table 1.

DMAA represents N,N-dimethylacrylamide, Y(NTf₂)₃ represents trifluoromethanesulfonimide yttrium, and Y(OTf)₃ represents yttrium trifluoromethanesulfonate. The conversion represents the percentage of polymerization of the monomer. The measurement device used was a ¹H-NMR device.

The binaphthol derivatives are compounds represented by the formula (2). The configuration [(R), (S), (rac)] and the number n of ethylene oxide units are shown in Table 1. In the table, la represents n=0, 1b represents n=1, and 1c represents n=2.

### Example 16

### [Living Radical Polymerization]

In a nitrogen atmosphere, a dry glass reaction container was charged with 1.1 mL of dichloromethane as a solvent, 0.57 mmol of N,N-diethylacrylamide (DEAA) as a monomer, 0.23 mmol (0.20 equivalents) of ytterbium trifluoromethanesulfonate [Yb(OTf)₃] as a Lewis acid, 0.23 mmol (0.20 equivalents) of water as a ligand, and 0.0057 mmol (0.20 equivalents) of an organotellurium compound represented by the formula (5) as a chain transfer agent. They were then stirred at room temperature for 30 minutes to prepare a polymerization reaction solution. The obtained polymerization reaction solution was polymerized at 0° C. for 10 hours while being irradiated with a 9.4-W LED lamp, whereby a polymer was obtained.

### Examples 17 to 39 and Comparative Examples 6 to 10

Polymers were obtained by polymerization as in Example 16, except that the polymerization conditions (temperature and time) were as shown in Table 2 and the types and amounts (equivalents) of monomers, Lewis acids, and ligands were as shown in Table 2. Specifically, Examples 17 to 39 and Comparative Examples 6 to 10 used the organotellurium compound as a chain transfer agent. NIPAM represents N-isopropylacrylamide. The conversion in Table 2 represents the percentage of polymerization of the monomer. The binaphthol derivatives are compounds represented by the formula (2). The configuration [(R), (S), (rac)] and the number n of ethylene oxide units are shown in Table 2. In the table, la represents n=0, 1b represents n=1, and 1c represents n=2.

### Evaluation Methods

The polymers obtained above were evaluated by the following methods. Tables 1 and 2 show the results. The results were verified taking the types of the monomers and the Lewis acids into consideration. The results of Comparative Examples 3 and 10 were shown as “-” (unmeasurable) because no polymer was formed.

(1) Molecular Weight Measurement (Mn and D)

The number average molecular weight (Mn), the weight average molecular weight (Mw), and the molecular weight distribution D (Mw/Mn) of each polymer were measured by size exclusion chromatography (SEC). The measurement was performed using Shodex LF-604 (polystyrene gel column) produced by Showa Denko K.K.

(2) ¹H-NMR Measurement

The percentage of a meso form in each polymer was measured by ¹H-NMR measurement. The ¹H-NMR measurement was performed at 130° C. using a solution in DMSO-d₆. The percentage of a meso form in a polymer chain serves as an index of stereoselectivity. A high percentage of a meso form is direct evidence of control of stereoselectivity.

TABLE 1

| | | | | | | | | | Evaluation | | |
|---|---|---|---|---|---|---|---|---|---|---|---|
| | Composition | | | | | Polymerization conditions | | | | | Meso form |
| | Monomer | Lewis acid | | Ligand | | Temperature | Time | Conversion | | | percentage |
| | type | Type | Equivalents | Type | Equivalents | (° C.) | (hr) | (%) | Mn | D | (%) |
| Example 1 | DEAA | Yb(OTf)₃ | 0.20 | H₂O | 0.20 | 0 | 14 | 97 | 41,700 | 3.9 | 94 |
| Example 2 | DEAA | Yb(OTf)₃ | 0.20 | CH₃OH | 0.20 | 0 | 14 | 89 | 25,000 | 4.1 | 94 |
| Example 3 | DEAA | Yb(OTf)₃ | 0.20 | (R)-1c | 0.20 | 0 | 14 | 78 | 18,300 | 2.3 | 94 |
| Example 4 | DEAA | Yb(OTf)₃ | 0.10 | (R)-1c | 0.10 | 0 | 14 | 96 | 2,700 | 3.2 | 88 |
| Example 5 | DEAA | Yb(OTf)₃ | 0.30 | (R)-1c | 0.30 | 0 | 14 | 67 | 7,800 | 1.6 | 91 |
| Example 6 | DEAA | Yb(OTf)₃ | 0.20 | (S)-1c | 0.20 | 0 | 14 | 79 | 17,200 | 1.9 | 94 |
| Example 7 | DEAA | Yb(OTf)₃ | 0.20 | (rac)-1c | 0.20 | 0 | 14 | 96 | 15,000 | 2.0 | 94 |
| Comparative | DEAA | None | — | None | — | 0 | 14 | 57 | 15,700 | 1.7 | 56 |
| Example 1 | | | | | | | | | | | |
| Comparative | DEAA | Yb(OTf)₃ | 0.20 | None | — | 0 | 14 | 99 | 16,900 | 1.9 | 84 |
| Example 2 | | | | | | | | | | | |
| Example 8 | DEAA | Y(NTf₂)₃ | 0.20 | (R)-1c | 0.20 | 0 | 14 | 95 | 4,000 | 1.53 | 86 |
| Example 9 | DEAA | Y(NTf₂)₃ | 0.20 | (rac)-1c | 0.20 | 0 | 14 | 98 | 4,100 | 1.53 | 86 |
| Comparative | DEAA | Y(NTf₂)₃ | 0.20 | None | — | 0 | 14 | 9 | — | — | — |
| Example 3 | | | | | | | | | | | |
| Example 10 | DMAA | Y(OTf)₃ | 0.20 | (R)-1c | 0.20 | 0 | 2 | 84 | 12,500 | 2 | 83 |
| Example 11 | DMAA | Y(OTf)₃ | 0.50 | H₂O | 0.50 | 0 | 2 | 45 | 14,700 | 1.8 | 72 |
| Example 12 | DMAA | Y(OTf)₃ | 0.50 | (R)-1a | 0.50 | 0 | 2 | 48 | 8,200 | 1.6 | 68 |
| Example 13 | DMAA | Y(OTf)₃ | 0.50 | (R)-1b | 0.50 | 0 | 2 | 95 | 9,800 | 2 | 78 |
| Example 14 | DMAA | Y(OTf)₃ | 0.50 | (R)-1c | 0.50 | 0 | 2 | 89 | 10,000 | 1.6 | 82 |
| Comparative | DMAA | Y(OTf)₃ | 0.20 | None | — | 0 | 2 | 55 | 5,400 | 1.6 | 62 |
| Example 4 | | | | | | | | | | | |
| Example 15 | DMAA | Yb(OTf)₃ | 0.20 | (R)-1c | 0.20 | 0 | 2 | 45 | 29,000 | 2.7 | 84 |
| Comparative | DMAA | None | — | None | — | 0 | 2 | 47 | 5,500 | 1.6 | 48 |
| Example 5 | | | | | | | | | | | |

TABLE 2

| | Composition | | | | | |
|---|---|---|---|---|---|---|
| | Monomer | Lewis acid | | Ligand | | Chain transfer |
| | type | Type | Equivalents | Type | Equivalents | agent |
| Example 16 | DEAA | Yb(OTf)₃ | 0.20 | H₂O | 0.20 | Organotellurium |
| Example 17 | DEAA | Yb(OTf)₃ | 0.20 | H₂O | 0.20 | compound |
| Example 18 | DEAA | Yb(OTf)₃ | 0.20 | CH₃OH | 0.20 | |
| Example 19 | DEAA | Yb(OTf)₃ | 0.20 | CH₃OH | 0.20 | |
| Example 20 | DEAA | Yb(OTf)₃ | 0.20 | CH₃OH | 0.40 | |
| Example 21 | DEAA | Yb(OTf)₃ | 0.20 | CH₃OH | 0.10 | |
| Example 22 | DEAA | Yb(OTf)₃ | 0.20 | CH₃OH | 0.10 | |
| Example 23 | DEAA | Yb(OTf)₃ | 0.20 | (CH₃)₂CHOH | 0.20 | |
| Example 24 | DEAA | Yb(OTf)₃ | 0.20 | (CH₃)₃COH | 0.20 | |
| Example 25 | DEAA | Yb(OTf)₃ | 0.20 | 4-BrC₆H₄OH | 0.20 | |
| Example 26 | DEAA | Yb(OTf)₃ | 0.20 | CH₃CH₂OH | 0.20 | |
| Example 27 | DEAA | Yb(OTf)₃ | 0.20 | HO(CH₂)₂OH | 0.20 | |
| Example 28 | DEAA | Yb(OTf)₃ | 0.20 | HO(CH₂)₃OH | 0.20 | |
| Example 29 | DEAA | Yb(OTf)₃ | 0.20 | HO(CH₂)₄OH | 0.20 | |
| Example 30 | DEAA | Yb(OTf)₃ | 0.20 | HOC(CH₃)₂CH₂CH(OH)CH₃ | 0.20 | |
| Example 31 | DEAA | Yb(OTf)₃ | 0.20 | HO(C₂H₄O)₃H | 0.20 | |
| Example 32 | DEAA | Yb(OTf)₃ | 0.20 | HO(C₂H₄O)₄H | 0.20 | |
| Example 33 | DEAA | Yb(OTf)₃ | 0.20 | CH₃OC₂H₄OH | 0.20 | |
| Example 34 | DEAA | Yb(OTf)₃ | 0.20 | CH₃O(C₂H₄O)₂H | 0.20 | |
| Example 35 | DEAA | Yb(OTf)₃ | 0.20 | (R)-1c | 0.20 | |
| Example 36 | NIPAM | Yb(OTf)₃ | 0.20 | H₂O | 0.20 | Organotellurium |
| Example 37 | NIPAM | Y(OTf)₃ | 0.10 | H₂O | 0.10 | compound |
| Example 38 | NIPAM | Y(OTf)₃ | 0.10 | CH₃OH | 0.10 | |
| Example 39 | NIPAM | Y(OTf)₃ | 0.10 | CH₃OH | 1.00 | |
| Comparative | DEAA | None | — | None | — | Organotellurium |
| Example 6 | | | | | | compound |
| Comparative | DEAA | Yb(OTf)₃ | 0.20 | None | — | |
| Example 7 | | | | | | |
| Comparative | DEAA | Yb(OTf)₃ | 0.20 | CH₃O(C₂H₄O)₂CH₃ | 0.20 | |
| Example 8 | | | | | | |
| Comparative | NIPAM | Yb(OTf)₃ | 0.20 | None | — | Organotellurium |
| Example 9 | | | | | | compound |
| Comparative | NIPAM | Y(OTf)₃ | 0.10 | None | — | |
| Example 10 | | | | | | |

| | | | | Evaluation | | |
|---|---|---|---|---|---|---|
| | Polymerization conditions | | | | | Meso form |
| | Temperature | Time | Conversion | | | percentage |
| | (° C.) | (hr) | (%) | Mn | D | (%) |
| Example 16 | 0 | 10 | 99 | 19,500 | 1.09 | 94 |
| Example 17 | 0 | 2 | 99 | 18,500 | 1.09 | 94 |
| Example 18 | 0 | 14 | 99 | 22,800 | 1.09 | 94 |
| Example 19 | 0 | 2 | 99 | 20,300 | 1.09 | 94 |
| Example 20 | 0 | 14 | 99 | 22,800 | 1.09 | 94 |
| Example 21 | 0 | 10 | 99 | 19,500 | 1.09 | 94 |
| Example 22 | 0 | 2 | 99 | 19,100 | 1.1 | 94 |
| Example 23 | 0 | 14 | 81 | 14,300 | 1.18 | 93 |
| Example 24 | 0 | 14 | 98 | 17,400 | 1.12 | 88 |
| Example 25 | 0 | 14 | 80 | 15,200 | 1.08 | 88 |
| Example 26 | 0 | 14 | 99 | 21,200 | 1.1 | 94 |
| Example 27 | 0 | 14 | 99 | 21,200 | 1.1 | 94 |
| Example 28 | 0 | 14 | 99 | 21,200 | 1.12 | 94 |
| Example 29 | 0 | 14 | 99 | 21,700 | 1.09 | 94 |
| Example 30 | 0 | 14 | 59 | 13,000 | 1.15 | 94 |
| Example 31 | 0 | 14 | 99 | 21,600 | 1.07 | 94 |
| Example 32 | 0 | 14 | 24 | 6,800 | 1.16 | 94 |
| Example 33 | 0 | 14 | 89 | 19,900 | 1.1 | 94 |
| Example 34 | 0 | 14 | 70 | 16,100 | 1.14 | 94 |
| Example 35 | 0 | 8 | 99 | 20,300 | 1.16 | 94 |
| Example 36 | 0 | 10 | 47 | 21,700 | 1.5 | 67 |
| Example 37 | 0 | 10 | 54 | 8,700 | 1.23 | 69 |
| Example 38 | 0 | 12 | 52 | 11,700 | 1.13 | 75 |
| Example 39 | 0 | 12 | 83 | 18,100 | 1.09 | 86 |
| Comparative | 25 | 70 | 90 | 11,000 | 1.04 | 56 |
| Example 6 | | | | | | |
| Comparative | 0 | 10 | 99 | 15,400 | 1.08 | 84 |
| Example 7 | | | | | | |
| Comparative | 0 | 14 | 90 | 20,400 | 1.11 | 84 |
| Example 8 | | | | | | |
| Comparative | 0 | 10 | 35 | 10,200 | 1.31 | 64 |
| Example 9 | | | | | | |
| Comparative | 0 | 10 | 4 | — | — | — |
| Example 10 | | | | | | |

### INDUSTRIAL APPLICABILITY

The present invention can provide a stereocontrol catalyst for use in radical polymerization that is applicable to polymerization of a broad range of monomers and that enables polymerization with control of both molecular weight (molecular weight distribution) and stereoselectivity, a method for producing a polymer using the stereocontrol catalyst for use in radical polymerization, and a polymer.

1. A stereocontrol catalyst for use in radical polymerization, comprising:
a rare-earth metal salt compound; and
a hydroxy group-containing compound.

2. The stereocontrol catalyst for use in radical polymerization according to claim 1, wherein the rare-earth metal salt compound is a rare-earth metal trifluoromethanesulfonate.

3. The stereocontrol catalyst for use in radical polymerization according to claim 1, wherein the rare-earth metal salt compound is a salt of a trivalent rare-earth metal.

4. The stereocontrol catalyst for use in radical polymerization according to claim 1, wherein the hydroxy group-containing compound is an alcoholic compound.

5. The stereocontrol catalyst for use in radical polymerization according to claim 1, wherein the hydroxy group-containing compound is a binaphthol derivative.

6. A method for producing a polymer, comprising:
polymerizing a (meth)acrylic monomer under the presence of the stereocontrol catalyst for use in radical polymerization according to claim 1.

7. The method for producing a polymer according to claim 6,
wherein the (meth)acrylic monomer is polymerized under the presence of the stereocontrol catalyst for use in radical polymerization in a solvent that contains a compound different from a hydroxy group-containing compound contained in the stereocontrol catalyst for use in radical polymerization.

8. The method for producing a polymer according to claim 6, which is performed by living radical polymerization.

9. An acrylic polymer comprising, in a molecule:
a meso form; and
a racemo form,
the acrylic polymer having a percentage of a meso portion of 65% or more and a molecular weight distribution of 1.8 or less.
